(19) United States
(12) Patent Application Publication
(10) Pub. No.: US 2016/0000693 A1
(43) Pub. Date: Jan. 7, 2016

(54) COMPOSITION COMPRISING A DICARBOXYLIC ACID AND AN OIL, AND HAIR STRAIGHTENING PROCESS

(71) Applicant: L'OREAL, Paris (FR)

(72) Inventor: Virginie BURCKBUCHLER, Paris (FR)

(73) Assignee: L'OREAL, Paris (FR)

(21) Appl. No.: 14/764,751

(22) PCT Filed: Jan. 29, 2014
(86) PCT No.: PCT/EP2014/051694

§ 371 Date: Jul. 30, 2015

(30) Foreign Application Priority Data
Jan. 31, 2013 (FR) 1350825

(51) Int. Cl. A61K 8/92; A61K 8/362; A61K 8/36; A61Q 5/04

(52) U.S. Cl. CPC A61K 8/922; A61Q 5/04; A61K 8/362; A61K 8/361

(57) ABSTRACT

The invention relates to a composition comprising, in a physiologically acceptable aqueous medium, a dicarboxylic acid containing from 2 to 8 carbon atoms, and a plant oil of triglyceride type with a fatty acid fraction comprising not more than 20% by weight of oleic acid, the composition having a pH ranging from 7 to 11. Process for straightening African-type hair using the composition and a straightening step with an iron at a temperature of at least 100° C.

The invention relates to a cosmetic composition comprising a plant oil and a dicarboxylic acid, and to the use thereof for straightening African-type frizzy hair.

Many people are dissatisfied with the appearance of their hair; in particular, people who have curly hair, for instance African-type frizzy hair, usually wish to acquire straight hair, and, conversely, people who have curl-free hair wish to have curly hair.

The first of the techniques usually used for permanently reshaping the hair consists, in a first step, in opening the —S—S— disulfide bonds of keratin (keratocystine) using a composition containing a suitable reducing agent (reduction step), and then, after having rinsed the head of hair thus treated, generally with water, in reconstituting said disulfide bonds, in a second step, by applying to the hair, which has been placed under tension beforehand with, for example, rollers, an oxidizing composition (oxidation step, also known as the fixing step) so as finally to give the hair the desired shape. This technique therefore allows the hair to be waved (permanent-waving procedure) and/or straightened (relaxing). The new shape given to the hair by a chemical treatment such as that above is long-lasting and in particular withstands washing with water or with shampoos, as opposed to the simple conventional techniques of temporary reshaping, such as hairsetting.

The reducing compositions that may be used for performing the first step of a permanent-reshaping operation, and in particular for straightening, generally contain sulfites, bisulfites, alkylphosphines or, preferably, thiols as reducing agents. Among the latter, those commonly used are cysteine and derivatives thereof, cysteamine and derivatives thereof, thiolactic acid or thioglycolic acid, and salts thereof and also esters thereof, especially glyceryl thioglycolate.

The oxidizing compositions required for performing the fixing step are usually compositions based on aqueous hydrogen peroxide solution.

In the context of hair relaxing and straightening techniques, this permanent reshaping operation is generally performed on curly or voluminous hair so as to obtain more or less pronounced straightening and a reduction of the volume and apparent mass of the hair.

However, such a technique is not yet entirely satisfactory. This is because, although this technique proves to be very effective for modifying the shape of the hair, it still degrades the hair fibers, which is mainly due to the high contents of reducing agents used in the reducing compositions and also to the various longer or shorter leave-on times that may be involved in such a process.

This technique can thus induce, in the long term, impairment of the quality of the hair, leading to a decrease in its cosmetic properties, such as its sheen, and degradation of its mechanical properties, more particularly of its mechanical strength, due to swelling of the hairs during the rinsing between the reduction step and the oxidation step, which can also be reflected by an increase in the porosity of the hairs. These drawbacks are in particular observed with thioglycolic acid, which is generally used in a basic medium at pH values between 8.5 and 9.5.

It has also been observed that the use of reducing agents results in an unsatisfactory durability for the straightening of the hair, in particular for the relaxing or defrizzing of the hair.

Finally, it is very common to have to deal with problems of odors, both with the reducing compositions used, and in particular those containing thiols, and with the hair reduced.

The second of the techniques normally used for obtaining hair straightening or relaxing consists in performing an operation known as lanthionization, using a composition containing a base belonging to the hydroxide family. It leads to replacement of the disulfide bonds (—CH₂—S—S—CH₂—) with lanthionine bonds (—CH₂—S—CH₂—). This lanthionization operation involves two consecutive chemical reactions:
The first reaction consists of a beta-elimination on the cystine caused by a hydroxide ion, resulting in the breaking of this bond and in the formation of dehydroalanine, as shown in the following reaction scheme.

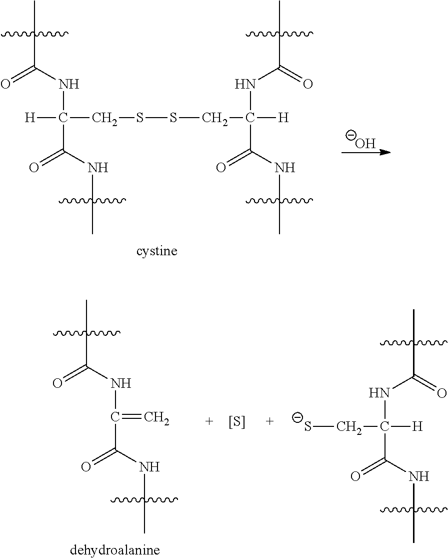

The second reaction is a reaction of the dehydroalanine with a thiol group. Specifically, the double bond of the dehydroalanine formed is a reactive double bond. It can react with the thiol group of the cysteine residue which has been freed so as to form a new bond known as a lanthionine bridge or bond or residue. This second reaction is illustrated by the following reaction scheme.

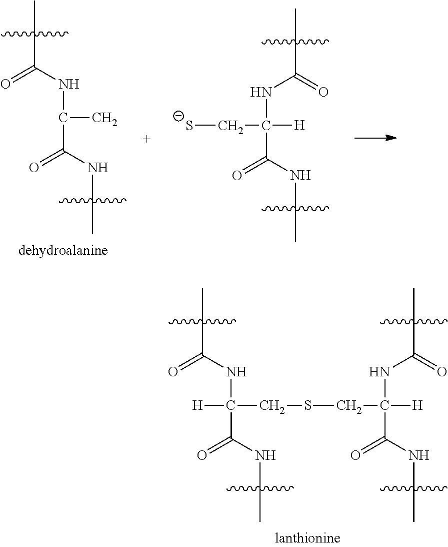

Compared with the first technique described above that uses a reducing agent, this lanthionization technique does not require a fixing step since the formation of the lanthionine bridges is irreversible.

It is therefore performed in a single step and makes it possible without distinction either to wave the hair, or to shape or defrizz or straighten the hair. This technique is mainly used for shaping naturally frizzy hair.

However, the hydroxides employed during this process have the major drawback of being caustic. This causticity affects the scalp by causing irritation which is sometimes severe, and can also affect the condition of the hair by making it, on the one hand, rough to the touch and, on the other hand, much more brittle. The use of hydroxides can also in certain cases cause bleaching of the natural color of the hair.

A hair relaxing process is known from document WO 2010/049 434, comprising the application to the hair of a composition comprising a dicarboxylic acid, followed by a heating step. However, the relaxing performance decreases rapidly over time, especially when the relaxed hair has been shampooed several times.

There is thus a need to improve the relaxing performance qualities on African-type frizzy hair of such a straightening/relaxing process, and in particular to maintain good straightening/relaxing over time, especially after shampooing several times.

The Applicant has demonstrated that by using a dicarboxylic acid with a particular plant oil as defined below, the relaxing performance qualities on African-type frizzy hair are improved, as is the long-lasting effect thereof over time, especially after shampooing several times.

Thus, one subject of the present invention is a composition comprising, in a physiologically acceptable aqueous medium, a dicarboxylic acid containing from 2 to 8 carbon atoms, and a plant oil of triglyceride type with a fatty acid fraction comprising not more than 20% by weight of oleic acid, the composition having a pH ranging from 7 to 11.

A subject of the invention is also a process for straightening African-type frizzy hair, comprising a step of applying to the African-type frizzy hair a composition as defined previously, followed by a step of straightening the hair using a straightening iron at a temperature of at least 100° C., preferably ranging from 100 to 250° C.

A subject of the invention is also the use of the composition as defined previously for straightening/relaxing African-type frizzy hair.

The composition according to the invention comprises an optionally hydroxylated dicarboxylic acid containing from 2 to 8 carbon atoms.

The dicarboxylic acid may be chosen from maleic acid, malic acid, itaconic acid, oxalic acid, malonic acid, oxomalonic acid, fumaric acid, succinic acid, tartaric acid, adipic acid, glutaric acid, α-ketoglutaric acid and galactaric acid, salts thereof, and mixtures thereof.

The salts may be chosen from the sodium, lithium, potassium and ammonium salts. The salts are preferably sodium salts.

Advantageously, the dicarboxylic acid may be chosen from maleic acid, malic acid, itaconic acid, adipic acid and glutaric acid, salts thereof, and mixtures thereof.

The salts may be chosen from the alkali metal salts, for example of sodium or potassium; or the alkaline-earth metal salts, for example of calcium, magnesium or strontium. The sodium salt is preferably used.

Maleic acid is preferably used. Maleic acid is used in particular in the form of the sodium salt.

Advantageously, the dicarboxylic acid may be present in the composition according to the invention in a content ranging from 0.5% to 10% by weight and preferably ranging from 1% to 5% by weight relative to the total weight of the composition.

The composition according to the invention comprises a plant oil whose triglycerides have a fatty acid fraction comprising not more than 20% by weight of oleic acid (C18:2).

Plant oils that may be used include avocado oil (oleic acid content ranging from 8 to 15), olive oil (oleic acid content ranging from 3.5 to 20) or coriander oil (oleic acid content ranging from 9 to 18).

Preferentially, the plant oil is avocado oil.

The plant oil used according to the invention may be present in the composition according to the invention in a content ranging from 1% to 50% by weight, preferably ranging from 3% to 40% by weight and preferentially ranging from 5% to 30% by weight relative to the total weight of the composition.

The composition according to the invention comprises water, especially in a content ranging from 10% to 95% by weight and preferably ranging from 20% to 95% by weight relative to the total weight of the composition.

The composition according to the invention contains a physiologically acceptable medium, i.e. a medium that is compatible with human keratin materials such as the skin (of the body, face, around the eyes or the scalp), the hair, the eyelashes, the eyebrows, bodily hair, the nails or the lips.

The composition according to the invention may comprise a physiologically acceptable organic solvent chosen, for example, from lower alcohols containing from 2 to 8 carbon atoms and in particular from 2 to 6 carbon atoms, for instance ethanol, isopropanol, propanol or butanol; polyethylene glycols containing from 6 to 80 ethylene oxide units, and polyols, for instance propylene glycol, isoprene glycol, butylene glycol, glycerol and sorbitol.

Advantageously, the aqueous medium of the composition according to the invention has a pH ranging from 7 to 11 and preferably ranging from 8 to 10.

The composition according to the invention may comprise a base. The base may be chosen from mineral bases such as LiOH, NaOH, KOH, Ca(OH)₂, NH₄OH or Zn(OH)₂ or from organic bases such as a primary, secondary or tertiary (C₁-C₆)alkylamine, especially triethylamine or butylamine. The primary, secondary or tertiary alkylamine may comprise one or more nitrogen and/or oxygen atoms and may thus comprise, for example, one or more alcohol functions; mention may be made especially of 2-amino-2-methylpropanol, triethanolamine, dimethylethanolamine, methylethanolamine and 2-dimethylaminopropanol. Mention may also be made of lysine or 3-(dimethylamino)propylamine.

According to a preferred embodiment of the invention, the composition may comprise a thickening polymer chosen from (meth)acryloyloxy(C₁-C₄)alkyltri(C₁-C₄)alkylammonium salt polymers and nonionic cellulose-based polymers.

The thickening polymer may be a (meth)acryloyloxy(C₁-C₄)alkyltri(C₁-C₄)alkylammonium salt polymer, preferentially an ethyltrimethylammonium methacrylate chloride polymer, chosen especially from homopolymers or copolymers with acrylamide.

Such a polymer makes it possible to obtain good hair cosmetic properties after relaxing it, in particular good properties of softness of feel.

The (meth)acryloyloxy(C₁-C₄)alkyltri(C₁-C₄)alkylammonium salt polymer may be crosslinked, especially with an olefinically unsaturated compound, in particular methylenebisacrylamide.

As ethyltrimethylammonium methacrylate chloride homopolymer, use may be made of those sold under the name Cosmedia® Ultragel 300 by the company Cognis or Salcare® SC 95 and Salcare® SC 96 by the company Ciba.

As acrylamide/ethyltrimethylammonium methacrylate chloride copolymer, use may be made of those sold under the name Salcare® SC 92 by the company BASF.

Use is preferably made of an ethyltrimethylammonium methacrylate chloride homopolymer (INCI name: Polyquaternium-37).

According to another embodiment variant of the composition according to the invention, the thickening polymer may be a nonionic cellulose-based polymer, chosen especially from (C₁-C₄)alkylcelluloses such as methylcelluloses and ethylcelluloses (for example Ethocel Standard 100 Premium from Dow Chemical); (poly)hydroxy(C₁-C₄)alkylcelluloses such as hydroxymethylcelluloses, hydroxyethylcelluloses (for example Natrosol 250 HHR sold by Aqualon) and hydroxypropylcelluloses (for example Klucel EF from Aqualon); (poly)hydroxy(C₁-C₄)alkyl(C₁-C₄)alkylcelluloses, in particular hydroxypropylmethylcelluloses (for example Methocel F4M from Dow Chemical), hydroxyethylmethylcelluloses, hydroxyethylethylcelluloses (for example Bermocoll E 481 FQ from Akzo Nobel) and hydroxybutylmethylcelluloses. Use is preferably made of (poly)hydroxy(C₁-C₄)alkyl(C₁-C₄)alkylcelluloses. Hydroxypropylmethylcellulose is preferentially used.

Advantageously, the thickening polymer may be present in the composition according to the invention in a content ranging from 0.5% to 10% by weight and preferably ranging from 1% to 5% by weight relative to the total weight of the composition.

The composition according to the invention may comprise additional fatty substances which may be present in liquid, semi-solid or solid form, for instance oils, waxes or gums.

Additional fatty substances that may be mentioned include C6-C16 hydrocarbons and in particular alkanes, fluoro oils of synthetic origin, fatty alcohols, fatty acid and/or fatty alcohol monoesters, nonsilicone waxes, silicones, fatty amines, fatty acids and compounds of ceramide type, and also mixtures of these various compounds.

It is recalled that, for the purposes of the invention, the fatty alcohols, fatty esters and fatty acids more particularly contain one or more linear or branched, saturated or unsaturated hydrocarbon-based groups comprising 6 to 30 carbon atoms, which are optionally substituted, in particular, with one or more (in particular 1 to 4) hydroxyl groups. If they are unsaturated, these compounds can comprise one to three conjugated or nonconjugated carbon-carbon double bonds.

The C6-C16 alkanes may be chosen from hexane and dodecane, and isoparaffins such as isohexadecane and isodecane.

Among the fluoro oils, mention may also be made of perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or else bromoperfluorooctyl sold under the name Foralkyl® by the company Atochem; nonafluoromethoxybutane and nonafluoroethoxyisobutane; perfluoromorpholine derivatives such as 4-trifluoromethyl perfluoromorpholine sold under the name PF 5052® by the company 3M.

The fatty alcohols that may be used in the cosmetic composition are saturated or unsaturated, and linear or branched, and contain from 6 to 30 carbon atoms and more particularly from 8 to 30 carbon atoms. Examples that may be mentioned include cetyl alcohol, stearyl alcohol and the mixture thereof (cetylstearyl alcohol), octyldodecanol, 2-butyloctanol, 2-hexyldecanol, 2-undecylpentadecanol, oleyl alcohol and linoleyl alcohol.

The waxes that may be used in the cosmetic composition are chosen especially from carnauba wax, candelilla wax, esparto wax, paraffin wax, ozokerite, plant waxes, for instance olive wax, rice wax, hydrogenated jojoba wax or the absolute waxes of flowers such as the essential wax of blackcurrant blossom sold by the company Bertin (France), animal waxes, for instance beeswaxes, or modified beeswaxes (cerabellina); other waxes or waxy starting materials that may be used according to the invention are especially marine waxes such as the product sold by the company Sophim under the reference M82, and polyethylene waxes or polyolefin waxes in general.

As regards the fatty acid and/or fatty alcohol monoesters, mention may be made especially of monoesters of saturated or unsaturated, linear or branched C1-C26 aliphatic monoacids and of saturated or unsaturated, linear or branched C1-C26 aliphatic monoalcohols.

Among the monoesters, mention may be made of dihydroabietyl behenate; octyl-dodecyl behenate; isocetyl behenate; cetyl lactate; C12-C15 alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononanoate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate. The fatty acids are more particularly chosen from myristic acid, palmitic acid, stearic acid, behenic acid, oleic acid, linoleic acid, linolenic acid and isostearic acid.

The composition according to the invention may also contain one or more additives chosen from nonionic, anionic and amphoteric surfactants, vitamins and provitamins including panthenol, water-soluble and liposoluble sunscreens, fillers and solid particles, for instance mineral and organic, colored or uncolored pigments, nacreous agents and opacifiers, glitter flakes, active particles, mineral fillers, dyes, sequestrants, plasticizers, solubilizers, thickeners, antioxidants, anti-foams, moisturizers, emollients, penetrants, fragrances and preserving agents.

The composition according to the invention may be in any galenical form conventionally used for application to the hair and in particular in the form of aqueous solutions, oil-in-water (O/W), water-in-oil (W/O) or multiple (triple: W/O/W or O/W/O) emulsions, or aqueous gels. These compositions are prepared according to the usual methods.

### Relaxing Process

The cosmetic composition according to the invention may be used on dry or wet hair, preferentially on wet hair, with or without a leave-on time.

After applying the cosmetic composition according to the invention and before raising the temperature of the hair by means of an iron, said composition may be left on for a period ranging from 5 to 60 minutes, preferably ranging from 5 to 15 minutes. The leave-on time may be effected under heat, and in particular under an occlusive system.

The cosmetic composition according to the invention is advantageously applied to the frizzy hair in an amount ranging from 0.1 to 10 grams and preferably from 0.2 to 5 grams of composition per gram of hair.

After application of the cosmetic composition according to the invention, the hair can be wrung out in order to remove the excess composition.

As explained previously, the process according to the invention comprises a step of straightening frizzy hair at a temperature of at least 100° C., preferably ranging from 100 to 250° C., which is performed by means of an iron, after application of the cosmetic composition according to the invention.

The heating step is necessary to optimize the effects of the process.

For the purposes of the present invention, the term “iron” means a device for heating the hair which brings said hair and the heating device into contact.

The end of the iron which comes into contact with the hair generally has two flat surfaces. These two surfaces may be made of metal. In particular, these two surfaces may be smooth or crimped.

The heating step may be performed by means of a straightening iron, a curling iron, a crimping iron or a steam iron. Preferably, the heating step is performed by means of a straightening iron.

As examples of irons that may be used in the straightening process according to the invention, mention may be made of any type of flat iron, and in particular, in a nonlimiting manner, those described in patents U.S. Pat. Nos. 5,957,140 and 5,046,516.

The iron may be applied by successive separate strokes lasting a few seconds or by gradual movement or sliding along the locks.

Preferably, the iron is applied in the process according to the invention by a continuous movement from the root to the tip, in one or more passes, in particular in two passes each lasting from 5 seconds to 1 minute.

The use of the iron during the process according to the invention provides the hair with dry heat and not with wet heat, which enables reshaping and in particular permanent straightening of the hair.

Preferably, the step of heating the hair is performed at a temperature ranging from 100 to 250° C., preferably from 190 to 220° C., better still from 200 to 215° C., and in particular at a temperature of 210° C., for a period of time which may range from 5 seconds to one hour and preferentially from 5 seconds to one minute.

The straightening process according to the invention may also comprise an additional step of pre-drying after the application of the cosmetic composition and before the step of heating the hair performed at a temperature ranging from 100 to 250° C. so as to prevent significant release of steam which might burn the hairdresser's hands and the person's scalp. The pre-drying step may be performed by means of a hairdryer or a drying hood or else by drying in the open air. After treating with the iron, the hair may be optionally rinsed or washed with a shampoo. The hair is then optionally dried by means of a hairdryer or a drying hood.

The straightening process according to the invention advantageously does not comprise the application of a reducing composition, neither before, nor during, nor after the application of the cosmetic composition according to the invention. In particular, the cosmetic composition according to the invention is preferably free of reducing agents.

For the purposes of the present invention, the expression “composition free of reducing agents” refers to a composition containing less than 1% by weight of reducing agents relative to the total weight of the composition, preferably a composition not containing any reducing agents.

According to one embodiment, the straightening process according to the invention comprises a step of applying a cosmetic composition containing a dicarboxylic acid containing from 2 to 8 carbon atoms, and a plant oil whose triglycerides have a fatty acid fraction comprising not more than 20% by weight of oleic acid, a step of performing one or more cosmetic treatments of frizzy hair, in particular a step of applying a shampoo to the hair, and a step of heating the hair by means of an iron after application of said cosmetic composition according to the invention, until the desired shape or shape intensity is obtained.

During the relaxing or defrizzing of the hair, the cosmetic composition is applied to the hair, preferably wet hair, and the hair is then subjected to mechanical reshaping making it possible to fix it in its new shape, by means of a hair straightening operation, with a large-toothed comb, with the back of a comb, by hand or with a brush.

The step of heating the hair is then performed at a temperature ranging from 100 to 250° C. by means of an iron, preferably a flat iron, as indicated previously.

The present invention also relates to a kit comprising a cosmetic composition containing a dicarboxylic acid containing from 2 to 8 carbon atoms, and a plant oil whose triglycerides have a fatty acid fraction comprising not more than 20% by weight of oleic acid, the composition being in a container;
and an iron producing a temperature of at least 100° C.

The term “container” means any receptacle or packaging suitable for containing the composition.

The examples that follow are given as illustrations of the present invention.

### Example 1

The relaxing performance qualities of several compositions were evaluated according to the following protocol:

Treatment protocol:

Apply 3 g of composition to a 2.7 g lock of hair.

Leave to stand for 15 minutes.

Remove the excess composition with a comb.

Blow-dry with a hairdryer.

Apply a straightening iron at 210° C. 10 times along the entire length of the lock of hair.

The tests were performed under the following conditions:
Type V African frizzy hair
after one or two treatments according to the protocol described previously
remanence of the straightening after shampooing 10 times (Ultra Doux from Garnier)

To evaluate the long-lasting (remanent) nature of the relaxing, the relaxed lock was washed 10 times consecutively with a shampoo (Ultra Doux from Gamier) and then dried at room temperature.

Test compositions:

Composition 1: Gel with avocado oil

| | |
|---|---|
| Avocado oil (Avocado Oil RBD STAB RE3 | 10% |
| from Jan Dekker) | |
| Disodium salt of maleic acid | 2% |
| Ethyltrimethylammonium methacrylate chloride | 4% |
| (Cosmedia ® Ultragel 300 from Cognis) | |
| Dimethylethanolamine | 2% |
| Glycerol | 1% |
| Water qs | 100% |

Composition 2: control gel

| | |
|---|---|
| Disodium salt of maleic acid | 2% |
| Ethyltrimethylammonium methacrylate chloride | 4% |
| (Cosmedia ® Ultragel 300 from Cognis) | |
| Dimethylethanolamine | 2% |
| Glycerol | 1% |
| Water qs | 100% |

Composition 3: control oil
100% Avocado oil (Avocado Oil RBD STAB RE3 from Jan Dekker)

For each treated lock, the softness of feel, the gloss and the straightening were evaluated globally. The long-lasting nature of the relaxing after shampooing 10 times was also evaluated.

The performance qualities were graded as follows:
− for a bad result
+ for a poor result
++ for a moderate result
+++ for a good result

The overall performance resulting from the mean of the results obtained for the softness of feel, sheen and straightening properties was then determined.

The following results were obtained:

### Results for the Relaxing of Type V African Frizzy Hair

| | Overall | | Long-lasting nature |
|---|---|---|---|
| | performance | Overall performance | of the relaxing after |
| | after | after | shampooing 10 |
| Composition | 1 treatment | 2 treatments | times |
| Composition 1 | ++ | +++ | +++ |
| (invention) | | | |
| Composition 2 | + | ++ | ++ |
| (control gel) | | | |
| Composition 3 | − | − | − |
| (avocado oil) | | | |

The results obtained show that the avocado oil alone (composition 3) applied to the hair does not modify the degree of frizziness.
Composition 1 according to the invention makes it possible to obtain good straightening of the hair after one treatment, the straightening being greater after the second treatment. Thus, the combination of the disodium salt of maleic acid with avocado oil makes it possible to obtain performance results in terms of straightening, softness and sheen, and also of relaxing remanence, which are better than those obtained by treating the hair with each of the ingredients (compositions 2 and 3).

### Example 2

The performance of composition 1 according to the invention was compared with that of similar compositions but containing an oil other than avocado oil, at the same concentration.

The various oils tested are apricot kernel oil, liquid petroleum jelly, pentaerythrityl tetraisostearate (ester 1) and propanediol dicaprylate (ester 2).

The following results were obtained:

| | | | | Performance | | |
|---|---|---|---|---|---|---|
| | Performance after | | | after shampooing | | |
| | 1 treatment | | | 10 times | | |
| Test oil | Straightness | Feel | Gloss | Straightness | Feel | Gloss |
| Avocado | +++ | +++ | +++ | +++ | +++ | +++ |
| oil | | | | | | |
| Ester 1 | ++ | +++ | ++ | − | +++ | ++ |
| Apricot | ++ | +++ | ++ | − | +++ | ++ |
| kernel oil | | | | | | |
| Ester 2 | ++ | ++ | ++ | ++ | ++ | ++ |
| Liquid | + | ++ | − | − | + | − |
| petroleum | | | | | | |
| jelly | | | | | | |

Among the oils evaluated, only avocado oil makes it possible to obtain the best results in terms of straightening, softness of feel and sheen after the treatment of the hair; it also makes it possible to obtain a good long-lasting effect of the relaxing after shampooing 10 times.

1-12. (canceled)

13. A composition comprising, in a physiologically acceptable aqueous medium:
a dicarboxylic acid containing from 2 to 8 carbon atoms, and
a plant oil of triglyceride type with a fatty acid fraction comprising not more than 20% by weight of oleic acid,
wherein the composition has a pH ranging from about 7 to about 11.

14. The composition according to claim 13, wherein the dicarboxylic acid is chosen from maleic acid, malic acid, itaconic acid, oxalic acid, malonic acid, oxomalonic acid, fumaric acid, succinic acid, tartaric acid, adipic acid, glutaric acid, α-ketoglutaric acid, or galactaric acid, salts thereof, or mixtures thereof.

15. The composition according to claim 13, wherein the dicarboxylic acid is maleic acid.

16. The composition according to claim 13, wherein the dicarboxylic acid is chosen from alkali metal salts or alkaline-earth metal salts of maleic acid, malic acid, itaconic acid, oxalic acid, malonic acid, oxomalonic acid, fumaric acid, succinic acid, tartaric acid, adipic acid, glutaric acid, α-ketoglutaric acid, or galactaric acid.

17. The composition according to claim 13, wherein the dicarboxylic acid is present in an amount ranging from about 0.5% to about 10% by weight, relative to the total weight of the composition.

18. The composition according to claim 13, wherein the dicarboxylic acid is present in an amount ranging from about 1% to about 5% by weight, relative to the total weight of the composition.

19. The composition according to claim 13, wherein the plant oil of triglyceride type with a fatty acid fraction comprising not more than 20% by weight of oleic acid is chosen from avocado oil, olive oil, or coriander oil.

20. The composition according to claim 13, wherein the plant oil of triglyceride type with a fatty acid fraction comprising not more than 20% by weight of oleic acid is avocado oil.

21. The composition according to claim 13, wherein the plant oil of triglyceride type with a fatty acid fraction comprising not more than 20% by weight of oleic acid is present in an amount ranging from about 1% to about 50% by weight, relative to the total weight of the composition.

22. The composition according to claim 13, wherein the plant oil of triglyceride type with a fatty acid fraction comprising not more than 20% by weight of oleic acid is present in an amount ranging from about 3% to about 40% by weight, relative to the total weight of the composition.

23. The composition according to claim 13, wherein the plant oil of triglyceride type with a fatty acid fraction comprising not more than 20% by weight of oleic acid is present in an amount ranging from about 5% to about 30% by weight, relative to the total weight of the composition.

24. The composition according to claim 13, further comprising a base.

25. The composition according to claim 13, further comprising a thickening polymer chosen from (meth)acryloyloxy(C₁-C₄)alkyltri(C₁-C₄)alkylammonium salt polymers, or nonionic cellulose-based polymers.

26. A process for straightening hair, the method comprising:
applying to the hair a composition comprising, in a physiologically acceptable aqueous medium:
a dicarboxylic acid containing from 2 to 8 carbon atoms, and
a plant oil of triglyceride type with a fatty acid fraction comprising not more than 20% by weight of oleic acid,
wherein the composition has a pH ranging from about 7 to about 11; and
straightening the hair using a curling iron at a temperature of at least about 100° C.

27. The process according to claim 26, wherein the curling iron is at a temperature ranging from about 100° C. to about 250° C.

28. A kit comprising:
a container comprising a cosmetic composition, wherein the cosmetic composition comprises, in a physiologically acceptable aqueous medium:
a dicarboxylic acid containing from 2 to 8 carbon atoms, and
a plant oil of triglyceride type with a fatty acid fraction comprising not more than 20% by weight of oleic acid; and
an iron configured to produce a temperature of at least about 100° C.
